(19) United States
(12) Patent Application Publication
(10) Pub. No.: US 2012/0061267 A1
(43) Pub. Date: Mar. 15, 2012

(54) MediTowel Anti-Fungal Wipe

(76) Inventor: Sally Villalobos, Montebello, CA (US)

(21) Appl. No.: 12/881,910

(22) Filed: Sep. 14, 2010

(51) Int. Cl. B65D 71/00; A61K 31/137; A61P 31/02; A61K 9/70

(52) U.S. Cl. 206/223; 424/411; 514/655

(57) ABSTRACT

Medicated single use, bilateral and bi-textured substrate uniformly impregnated with medicated anhydrous OTC (over-the-counter) solution for the topical use of aseptically delivering a single dose of medication to the surface of the skin. Medicate solution and substrate is contained encapsulated within a heat sealed foil enclosure that is stable for at least five years.

### CROSS-REFERENCE TO RELATED APPLICATION

### STATEMENT REGARDING FEDERALLY SPONSORED RESEARCH OR DEVELOPMENT

Not applicable

### BACKGROUND OF THE INVENTION

1. Field of the Invention

The invention relates to a medicated antifungal towelette. More particularly, it relates to a pre-packaged single use disposable towelette/wipe that contains a full dose of an antifungal medication in liquid form.

2. Description of Related Art Including Information Disclosed Under 37 CFR 1.97

The current medicated treatment of athlete's foot or any type of fungal infection is done either by a medicated cream, spray, shower/bath product or even rubbing alcohol. The most effective ways are the medicated creams which can be prescribed to a person or over the counter creams and sprays found at a local drug store.

The continuous use of medicated antifungal creams is prone to contamination. Once the tube or other delivery system is open and continually exposed to air, bacteria, fungi and other organisms found in dirty hands and nails; may enter and contaminate the tube or other delivery system. Another flaw is the inconsistent application of the products. A person without measuring it correctly may put too much or too little of the required dose which may already have been weakened by contamination.

The current commercial method of treating fungal infections is to rub or spray the surface of the skin. The current antifungal medicine in the marketplace fails to attack the root of the infection.

A solution is proposed which incorporates the use of a disposable single use bi-textured substrate that contains a single medicated dose of an antifungal composition (“Antifungal Wipe”). The antifungal towelette/wipe is enclosed in an impermeable package that can maintain the wipes potency for at least 5 years.

The following listed patents further illustrate less relevant pre-packaged sheet materials saturated with medicaments: U.S. Pat. No. 2,402,982 to Steenbergen (bandage in petrolatum): U.S. Pat. No. 2,999,265 to Duane et al. (hermetically sealed pad impregnated with epidermal bacteriostatic agent, humectant and emollient): U.S. Pat. No. 3,783,869 to Schnipper (medicated sanitary napkin).

Terbinafine Hydrochloride is a well-known active ingredient in anti-fungal medication: U.S. Pat. No. 11,023,131 to Ishiduka (Antifungal agent for topical use): U.S. Pat. No. 4,921,844 to Patchett (Fungicidal Compositions and Methods).

Although the use of terbinafine hydrochlorideis well known as an antifungal agent, a need still exist for a portable and effective way to stop fungal infections. An antifungal wipe encased in an impermeable package can solve the athlete's foot issues US soldiers' abroad face when deployed in remote locations. A spray is encumbering and does not hold the potency a wipe may in prolonged deployments or storage.

### BRIEF SUMMARY OF THE INVENTION

The present invention is focused on a pre-packaged anti-fungal skin towelette/wipe. The wipe is manufactured as a bi-textured substrate uniformly impregnated with medicated anhydrous medicated solution. Stability studies show that the substrate, which as a product, is enclosed in a package will hold the wipes potency and moisture for over 5 years. In use, the wipe is a portable single use wipe that provides the user with a single uncontaminated dose of the anti-fungal medication. The wipe is rubbed on the skin to exfoliate ad expel any excess skin so that the medication may treat the fungal infection directly. The infection is treated and the wipe is thrown away.

### BRIEF DESCRIPTION OF THE DRAWINGS

FIG. 1 is an perspective view of the package enclosing the wipes

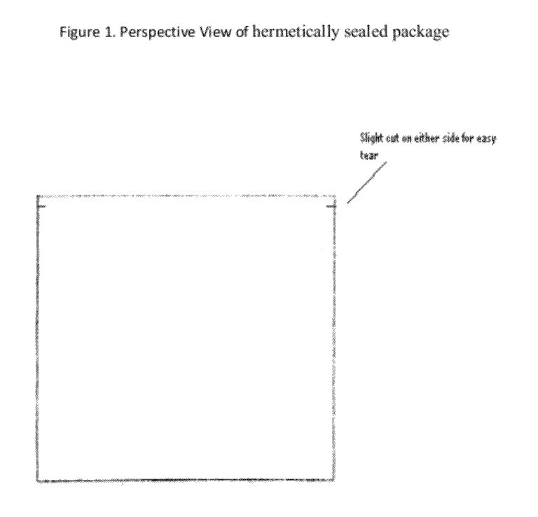

FIG. 2 is an environmental view of the side of the towelette with abrasions

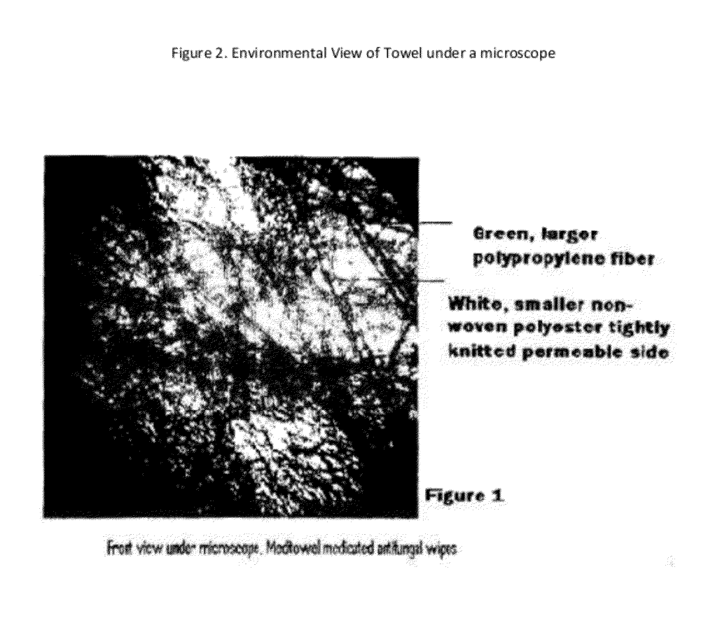

### DETAILED DESCRIPTION OF THE INVENTION

Anti-fungal medicines have been in the market for some time, they run from anti-fugal spray cans to creams. However, none of these wipes provide the portability and effectiveness of this invention. Anti-fungal cream or gel can dehydrate and become contaminated and lose its potency over time. When a bottle is used, it is exposed to air and bacteria. When the user approaches the end of a bottle, the bottle has been used and exposed countless times. In applying the medicine the user must “eyeball” the amount to apply on the infected area, thereby applying either too much or little of the medicine. Anti-fungal sprays also approach the same problems. Spraying the infected area only affects the fungus that sits on the surface and is difficult to spray in the nooks and crannies between the toes or between the skin and nail bed crevices. The amount sprayed also varies in amount where most of the powder residue dries and flakes off or attaches its self to socks or other feet coverings. A user may spray less than the recommended dose or more than is needed. The cans must also be kept at proper temperatures ranging from 32° F. to 120° F., which prevent storage and use problems in regards to portability.

The invention solves these problems in a unique way. The invention and the marketed product is a substrate containing an anhydrous treatment solution. The substrate used in the present invention comprises synthetic fiber in a non-woven, woven of knitted form. Of particular interest for use in the substrate employed in the present invention are the following fibers: polypropylene, polyester and rayon.

The substrate comprises synthetic, woven, non-woven or knitted fibers, or blends thereof. One side of the substrate contains larger fibers that create an abrasion. The abrasion is used to scrub and exfoliate the skin so that the anhydrous solution may penetrate the skin and expedite efficacy.

In accordance with the invention the anhydrous treatment solution contains terbinafine hydrochloride as the antifungal active. 1% of the active is found in the formula.

Terbinafine is characterized in its strong antifungal activity particular against the fungi dermatophytes such as Trichophyton, Microsporum and Epirdermophyton, as well as Malassezia, ringworm and jock itch. Terbinafine hydrochloride at a low concentration clearly exhibits a fungicidal effect on sterilizing germinated conidium of Trichophyton mentagrophytes (Schuster, I., et al.: “Preclinical characteristics of allylamines”; in Berg, D., Et al. eds. Sterol Biosynthesis inhibitors: Pharmaceutical and Agrochemical Aspects.: Pbl.: Ellis Horwood Ltd. Chichester (UK) pp. 449-470, 1998.

As for the mechanism of the function of Terbinafine hydrochloride, it selectively inhibits squalene epoxidase in the cells of fungi. It also causes the accumulation of squalene and reduction of ergosterol content and exhibits the antifungal effect. For dermatophytes, it destroys, even in a low concentration, the cell membrane formation to exhibit the fungicidal effect on the fungi.

Other ingredients found in the product include or may include: Propylene Glycol, SD Alcohol, Glycerin, Menthol Crystals, Peppermint Oil, Eucalyptus Oil, Rosemary Oil, Lavender Oil, Tea Tree Oil, Methylparaben, Propylparaben, and Fragrance.

The use of propylene glycol instead of water as a solvent is essential. Propylene glycol does not precipitate the release of free iodine, and thereby depletes its effectiveness before its actual use. Propylene glycol, unlike water, actually does preserve the stability of Terbinafine hydrochloride and facilitates an extended shelf life of the treated towelette/wipe. Propylene glycol is a lubricious emollient imparting soothing and softening qualities of the skin. Propylene glycol does not freeze in cold weather. The use of propylene glycol, as a non-aqueous solvent obviates the need for buffers, stabilizers, and preservatives which are generally required to be used in aqueous solutions.

A small amount of Alcohol not only has antimicrobial properties but also aids to solubilize the terbinafine hydrochloride.

Glycerin aids in moisturizing the cracked broken skin resulting from fungal infection and help to restore the healthy look and feel of the feet without introducing water which may contraindicate and become a living environment for fungi.

Menthol Crystals a crystalline alcohol form that is used as the cooling agent on the skin to alleviate the burning hot sensation caused by fungal infections on the surface of the skin.

Peppermint Oil (menthe piperita) is a strong volatile natural oil that has been used for centuries for its excellent antiseptic and anodyne properties. Also used as the “fragrance” in the product.

Eucalyptus Oil (eucalyptus globules) is a natural stimulating, antibacterial oil that is traditionally used as an antiseptic.

Rosemary Oil (rosmarinus officinalis) is another natural ingredient with antiseptic properties also known for its conditioning effects on the skin.

Lavender Oil is a mild antiseptic and natural fragrance oil.

Tea Tree Oil (melaleuca alternifolia) distilled from the leaves of the Australian tree is known to be the natural fungicide because of its antiseptic and germicidal properties.

Although the formula is stable and self preserved, a small amount of Methylparaben & Propylparaben preservatives are used to secure longevity and stability in the product.

The package of the wipe is a film that creates barrier vehicle whose properties not only contain the solution and substrate but prevent evaporation. Suitable and stable barrier materials are laminates, where a layer of ethylene vinyl alcohol or propylene vinyl alcohol or aluminum is sandwiched between polymeric film layers, such as low density polyethylene. Further suitable barrier materials for forming the package for the cleaning wipe herein are metallized, particularly aluminized plastics films; metal foils; oriented polyethylene terephthalate (PET); glycolmodified PET; oriented polyamidel; or polymeric films. Basically every material known in the art for this purpose is suitable for use herein. A very suitable laminate material for this purpose is for instance disclosed in EP 696,991.

The package of the wipes comprises opening aids for facilitating opening of the package. Exemplary opening aids are tear tapes, cuts or the like.

What is claimed is:

1. A packaged antifungal towelette, compromising:
A disposable, absorbent, sheet material suitable in size and strength for single-use wiping of the feet and body;
A Terbinafine Hydrochloride solution impregnated in said disposable, absorbent sheet material for providing antifungal relief to said sheet material; and,
A hermetically sealed envelope retaining said disposable, absorbent sheet-material.

2. A packaged antifungal towelette according to claim 1, wherein said hermetically sealed package is a tear-open packet.

3. A Terbinafine Hydrochloride solution according to claim 1, wherein said solution consist of 1% Terbinafine Hydrochloride and a combination of Propylene Glycol, SD Alcohol, Fragrance, Oils, and other ingredients

4. A disposable, absorbent sheet material according to claim 1, wherein said sheet material comprises of two sides, one side containing an abrasive surface.

5. An abrasive surface according to claim 4, wherein said abrasive surface is created by a layer of fiber on top of the sheet creating ridges.
